SEQUENCE LISTING (3 sequences)

SEQ ID NO: 1 - DNA, 23 bp, Artificial Sequence
  synthetic
cgaaggttaatctgtctccatct

SEQ ID NO: 2 - DNA, 28 bp, Artificial Sequence
  synthetic
cccaattaaaatttatataccaccacct

SEQ ID NO: 3 - DNA, 155 bp, Ommastrephes bartramii
cgaaggttaatctgtctccatcttattttttagaagttaatttttaaagtgaaaaagcttaaatctttcaaagggacgagaagaccctaatgagcttataattttattatcattatttaaaacatttaggtggtggtatataaattttaattggg

(19) United States
(12) Patent Application Publication
(10) Pub. No.: US 2021/0371903 A1
(43) Pub. Date: Dec. 2, 2021

(54) PCR PRIMER PAIR FOR IDENTIFICATION OF OMMASTREPHES BARTRAMII IN THE NORTH PACIFIC OCEAN BASED ON ENVIRONMENTAL DNA, AND METHOD FOR IDENTIFYING OMMASTREPHES BARTRAMII USING THE SAME

(71) Applicant: SHANGHAI OCEAN UNIVERSITY, Shanghai (CN)

(72) Inventors: Xinjun CHEN, Shanghai (CN); Qianqian WU, Shanghai (CN); Yang LIU, Shanghai (CN); Bilin LIU, Shanghai (CN); Congcong WANG, Shanghai (CN)

(21) Appl. No.: 17/147,649

(22) Filed: Jan. 13, 2021

(30) Foreign Application Priority Data
Jun. 2, 2020 (CN) 202010490062.0

(51) Int. Cl. C12Q 1/686; C12Q 1/6853

(52) U.S. Cl. CPC C12Q 1/686; C12Q 1/6853

(57) ABSTRACT

A primer pair for identification of Ommastrephes bartramii in the North Pacific Ocean based on environmental DNA, and a method for identifying Ommastrephes bartramii using the same. The primer pair consists of a forward primer OMBA-F: 5′-CGAAGGTTAATCTGTCTCCATCT-3′ (SEQ ID NO: 1), and a reverse primer OMBA-R: 5′-CCCAATTAAAATTTATATACCACCACCT-3′ (SEQ ID NO: 2). A DNA molecular marker locus for identifying Ommastrephes bartramii is located at 16S rRNA of Ommastrephes bartramii. The environmental DNA is amplified and sequenced, and if the obtained sequence is completely identical to the sequence of the DNA molecular marker locus, it is indicated that there is Ommastrephes bartramii in the sampling region.

### CROSS-REFERENCE TO RELATED APPLICATIONS

This application claims the benefit of priority from Chinese Patent Application No. 202010490062.0, filed on Jun. 2, 2020. The content of the aforementioned application, including any intervening amendments thereto, is incorporated herein by reference in its entirety.

### TECHNICAL FIELD

This disclosure relates to biotechnologies, and more particularly to a polymerase chain reaction (PCR) primer pair for identification of Ommastrephes bartramii in the North Pacific Ocean based on environmental DNA, and a method for identifying Ommastrephes bartramii using the same.

### BACKGROUND

Ommastrephes bartramii, a cephalopod with a life cycle of 1 year, is one of the most important oceanic economic fish species in the world and widely distributed in the North Pacific Ocean. There are mainly two breeding populations of the Ommastrephes bartramii: autumn cohort and winter-spring cohort, where the commercially harvested Ommastrephes bartramii in China is dominated by the west winter-spring cohort. Currently, the detection of Ommastrephes bartramii mainly relies on traditional fishing, which is time-consuming and laborious, also causes serious damages to the entire ecosystem. For multicellular organisms such as aquatic organisms, their metabolic wastes, damaged tissues and exfoliated skin tissues will remain in the water environment, and these tissues generally contain DNA information of the species (defined as environmental DNA, eDNA). With the help of the environmental DNA technology, an aquatic vertebrate (invasive Lithobates catesbeianus) was successfully identified from the water environment for the first time in 2008. Henceforth, the environmental DNA technology is gradually applied to the prevention and control of invasive species, protection of rare and endangered species, and ecological surveys of aquatic organisms.

The environmental DNA shed from the organism is broken into short fragments under the influence of environmental factors to remain in the environment, thus it is required to find a short species-specific gene (about 200 bp) to effectively detect a single fish species. At the same time, only a stable mutation locus in the gene that can distinguish the fish species from its closely-related species can be used as a molecular marker locus for the species identification based on environmental DNA. Besides the Ommastrephes bartramii, Todarodes pacificus and Heterololigo bleekeri are also distributed in the North Pacific Ocean, so it is required to design a molecular marker locus that can distinguish Ommastrephes bartramii from the Todarodes pacificus and Heterololigo bleekeri and a corresponding primer for efficiently conducting the ecological surveys of the Ommastrephes bartramii.

### SUMMARY

An object of this disclosure is to provide a PCR primer pair for identification of Ommastrephes bartramii in the North Pacific Ocean based on environmental DNA and a method for identifying Ommastrephes bartramii in the North Pacific Ocean using the same

The technical solutions of this disclosure are specifically shown as follows.

In a first aspect, this disclosure provides a PCR primer pair for identification of Ommastrephes bartramii in the North Pacific Ocean based on environmental DNA, comprising:

a forward primer OMBA-F:
(SEQ ID NO: 1)
5′-CGAAGGTTAATCTGTCTCCATCT-3′;
and
a reverse primer OMBA-R:
(SEQ ID NO: 2)
5′-CCCAATTAAAATTTATATACCACCACCT-3′.

In a second aspect, this disclosure further provides a method for identifying Ommastrephes bartramii in a region of the North Pacific by detecting environmental DNA with the primer pair, comprising:

(1) subjecting the environmental DNA to PCR amplification using the primer pair to obtain an amplification product; and

(2) comparing the amplification product with a sequence of a DNA molecular marker locus to determine whether there is Ommastrephes bartramii in the region;

wherein if the amplification product is identical to the sequence of the DNA molecular marker locus, it is indicated that there is Ommastrephes bartramii in the region of the North Pacific;

the DNA molecular marker locus is located on 16S rRNA of the Ommastrephes bartramii; and

the sequence of the DNA molecular marker locus is shown in SEQ ID NO: 3 with a length of 155 bp.

In an embodiment, the environmental DNA is obtained through steps of:

(a) collecting a water sample from the region of the North Pacific; filtering the water sample with a nitrocellulose filter with a diameter of 47 mm and a pore size of 0.4 μm; and storing the nitrocellulose filter at −20° C. for use; and

(b) extracting the environmental DNA from the nitrocellulose filter with a DNeasy Blood & Tissue kit (Qiagen); and storing the environmental DNA at −20° C. for use.

In an embodiment, the step (2) comprises:

subjecting the amplification product to electrophoresis using 2% agarose gel to obtain a positive band;

subjecting the positive band to DNA sequencing to obtain a sequence of the environmental DNA; and

comparing the sequence of the environmental DNA with the sequence of the DNA molecular marker locus to determine whether there is Ommastrephes bartramii in the region.

In some embodiments, a system of the PCR amplification has a volume of 20 μL and consists of:

2 μL of the environmental DNA as template;

15 μL of a PCR Mix;

1 μL of 10 μM forward primer OMBA-F;

1 μL of 10 μM reverse primer OMBA-R; and

ultrapure water.

In some embodiments, the PCR amplification is programmed as follows:

50° C. for 2 min;

95° C. for 10 min; and

55 cycles, each consisting of 95° C. for 15 s and 60° C. for 1 min.

The beneficial effects of this disclosure are shown as follows.
(1) This disclosure provides a DNA molecular marker locus of Ommastrephes bartramii and a PCR primer for amplifying the same, which can be used to effectively distinguish the Ommastrephes bartramii from other species distributed in the North Pacific Ocean.

(2) This disclosure provides a sampling and analysis method of environmental DNA, which can effectively avoid the pollution of environmental DNA samples and the degradation of the environmental DNA.

(3) In the identification method provided herein, it is only required to collect a water sample, extract an environmental DNA and perform sequence comparison to determine whether there is Ommastrephes bartramii in the region to be detected. Compared with traditional investigation methods, the method of the disclosure can avoid causing damage to the ecosystem and improve the detection accuracy.

### BRIEF DESCRIPTION OF THE DRAWINGS

FIG. 1 is an electrophoresis diagram showing products of PCR amplification of DNA from muscle tissues of Todarodes pacificus (1), Heterololigo bleekeri (2) and Ommastrephes bartramii (3) using a primer pair provided herein.

FIG. 2 is an electrophoresis diagram of a PCR amplification product of an environmental DNA from a water sample to be tested (1: blank control, 2 and 3: environmental DNA).

### DETAILED DESCRIPTION OF EMBODIMENTS

This disclosure provides a PCR primer pair for identifying Ommastrephes bartramii in the North Pacific Ocean based on environmental DNA, and a method for identifying Ommastrephes bartramii using the same. Specifically, a specific molecular marker is selected to effectively distinguish the Ommastrephes bartramii distributed in the North Pacific from two closely related species Todarodes pacificus and Heterololigo bleekeri. The specific molecular marker is expected to be suitable only for the identification of Ommastrephes bartramii, so as to specifically detect the Ommastrephes bartramii.

This disclosure will be fully described below with reference to the embodiments.

### Example 1

The validity of a PCR primer pair for identification of Ommastrephes bartramii was specifically analyzed as follows.

(1) DNA samples were separately extracted from muscle tissues of Ommastrephes bartramii and its two closely related species Todarodes pacificus and Heterololigo bleekeri with a DNeasy Blood & Tissue kit (Qiagen). After measured for the concentration, each DNA sample was diluted to 100 pg/μL for PCR amplification.

(2) Each diluted DNA sample obtained in step (1) was subjected to PCR amplification, where the PCR amplification system had a total volume of 20 μl and consisted of 2 μL of a template DNA, 15 μL of a PCR Mix, 1 μL of 10 μM OMBA-F, 1 μL of 10 μM OMBA-R and ultrapure water; and the PCR amplification was programmed as follows: 50° C. for 2 min; 95° C. for 10 min; and 55 cycles, each consisting of 95° C. for 15 s and 60° C. for 1 min.

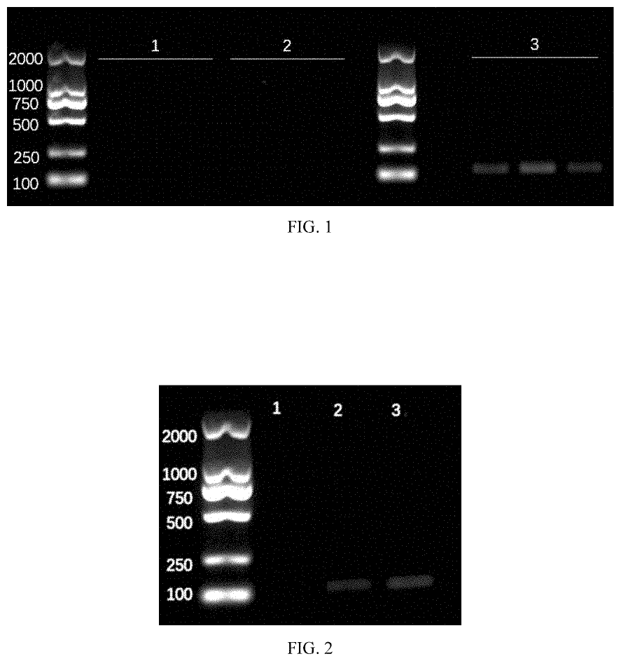

(3) PCR amplification products obtained in step (2) were detected by agarose gel (2%) electrophoresis, and the results were shown in FIG. 1. It was demonstrated that there was a fragment of about 155 bp in the amplification product of the DNA from Ommastrephes bartramii, while this fragment was absent in the amplification products of DNA samples from the two closely related species. The band appearing on the agarose gel was recovered and sequenced. The sequencing results confirmed that the target fragment can only be amplified from the DNA of Ommastrephes bartramii using the primer pair provided herein, that was, the primer pair provided herein can only be used to amplify the DNA of Ommastrephes bartramii, and failed to amplify the DNA of the two closely-related species. As demonstrated by the sequencing results, the amplification product was completely identical to the DNA sequence of Ommastrephes bartramii.

### Example 2

The identification of Ommastrephes bartramii in the North Pacific Ocean based on environmental DNA was performed as follows.

(1) 2 L of a surface water sample was collected by a water collector and filtered under vacuum with a nitrocellulose filter with a diameter of 47 mm and a pore size of 0.4 μm. The nitrocellulose filter was collected, folded in half and wrapped with tin foil. After the sample information was recorded, the filter was stored at −20° C. for use.

It was worth noting in the above process that a pair of disposable gloves should be worn before collecting the water sample; the sampling bottle should be washed 3 times with the water at the sampling site; the gloves should be replaced when the sampling site changed; a benzalkonium chloride reagent should be introduced to the water sample at a final concentration of 0.01% to prevent DNA degradation; it was required to shake the sampling bottle up and down about 5 times to mix the benzalkonium chloride with the water sample evenly; and direct sunlight should be avoided.

(2) The nitrocellulose filter collected in step (1) was treated with a DNeasy Blood & Tissue kit (Qiagen) to extract an environmental DNA, and the extracted environmental DNA was frozen and stored at −20° C.

(3) The environmental DNA obtained in step (2) was amplified by PCR using the primers OMBA-F and OMBA-R, where the PCR system had a total volume of 20 μL and consisted of 2 μL of the environmental DNA (as a template), 15 μL of a PCR Mix, 1 μL of 10 μM OMBA-F, 1 μL of 10 μM OMBA-R and ultrapure water; and the PCR amplification was programmed as follows: 50° C. for 2 min; 95° C. for 10 min; and 55 cycles, each consisting of 95° C. for 15 s and 60° C. for 1 min.

With regard to the PCR amplification, it should be noted that the volume of the DNA template can be adjusted according to its concentration; the number of cycles should not be less than 40 since the DNA of the target species had a low content in the environmental DNA; and the cross-contamination should be avoided when multiple samples were simultaneously subjected to PCR amplification.

(4) The amplified product obtained in step (3) was detected by agarose gel (2%) electrophoresis, and the results were shown in FIG. 2, from which a 155 bp positive band was observed. Then the positive band was recovered and sequenced. As demonstrated by the sequencing results, the amplification product was completely identical to the sequence of the DNA molecular marker locus of Ommastrephes bartramii, which indicated that there was Ommastrephes bartramii in the sampling region.

The above-mentioned embodiments are merely illustrative, and not intended to limit the invention. Any improvements, changes and modifications made by those skilled in the art without sparing any creative effort should fall within the scope of the invention defined by the appended claims.

What is claimed is:

1. A PCR primer pair for identification of Ommastrephes bartramii in the North Pacific Ocean based on environmental DNA, comprising:
a forward primer OMBA-F:
(SEQ ID NO: 1)
5′-CGAAGGTTAATCTGTCTCCATCT-3′;
and
a reverse primer OMBA-R:
(SEQ ID NO: 2)
5′-CCCAATTAAAATTTATATACCACCACCT-3′.

2. A method for identifying Ommastrephes bartramii in a region of the North Pacific based on environmental DNA using the primer pair of claim 1, comprising:
(1) subjecting the environmental DNA to PCR amplification using the primer pair to obtain an amplification product; and
(2) comparing the amplification product with a sequence of a DNA molecular marker locus to determine whether there is Ommastrephes bartramii in the region;
wherein if the amplification product is identical to the sequence of the DNA molecular marker locus, it is indicated that there is Ommastrephes bartramii in the region of the North Pacific Ocean;
the DNA molecular marker locus is located on 16S rRNA of the Ommastrephes bartramii; and
the sequence of the DNA molecular marker locus is shown in SEQ ID NO: 3 with a length of 155 bp.

3. The method of claim 2, wherein the environmental DNA is obtained through steps of:
(a) collecting a water sample from the region of the North Pacific Ocean;
filtering the water sample with a nitrocellulose filter with a diameter of 47 mm and a pore size of 0.4 μm; and storing the nitrocellulose filter at −20° C. for use; and
(b) extracting the environmental DNA from the nitrocellulose filter with a DNeasy Blood & Tissue kit (Qiagen); and storing the environmental DNA at −20° C. for use.

4. The method of claim 2, wherein the step (2) comprises:
subjecting the amplification product to electrophoresis with 2% agarose gel to obtain a positive band;
subjecting the positive band to DNA sequencing to obtain a sequence of the environmental DNA; and
comparing the sequence of the environmental DNA with the sequence of the DNA molecular marker locus to determine whether there is Ommastrephes bartramii in the region.

5. The method of claim 2, wherein a system of the PCR amplification has a volume of 20 μL and consists of:
2 μL of the environmental DNA as template;
15 μL of a PCR Mix;
1 μL of 10 μM forward primer OMBA-F;
1 μL of 10 μM reverse primer OMBA-R; and
ultrapure water.

6. The method of claim 2, wherein the PCR amplification is programmed as follows:
50° C. for 2 min;
95° C. for 10 min; and
55 cycles, each consisting of 95° C. for 15 s and 60° C. for 1 min.
